(19) United States
(12) Patent Application Publication
(10) Pub. No.: US 2002/0147482 A1
(43) Pub. Date: Oct. 10, 2002

(54) Wearable hemorrhoidal aid

(76) Inventor: Elijah Carter, Boulder City, NV (US)

Correspondence Address: Stephen M. De Klerk, Blakely, Sokoloff, Taylor & Zafman LLP, Seventh Floor, 12400 Wilshire Boulevard, Los Angeles, CA, 90025-1026, US

(21) Appl. No.: 09/827,387

(22) Filed: Apr. 4, 2001

(51) Int. Cl. A61F 7/00; A61F 13/00

(52) U.S. Cl. 607/108; 607/114; 602/67

(57) ABSTRACT

The invention provides a wearable hemorrhoidal aid, which includes a waistband, a crotch portion, and a rear portion. The waistband forms a closed loop. The crotch portion has a front edge attached to a front section of the waistband. The rear portion has a front edge attached to a rear edge of the crotch portion and a rear edge attached to a rear edge of the waistband. The rear portion includes a pouch defining an enclosure. The pouch has an opening for insertion of a thermal insert into the enclosure. The thermal insert is removable from the pouch. The insert may be preheated or precooled. The rear portion will then provide heating or cooling to an area near the anus where hemorrhoids may occur.

### BACKGROUND OF THE INVENTION

1). Field of the Invention

This invention relates to a wearable hemorrhoidal aid.

2). Discussion of Related Art

Hemorrhoids can often effectively be treated by heating or cooling with a thermal pad. A thermal pad including a shell and a substance located within the shell may be located in a microwave or located in a refrigerator to either heat or cool the pad and then be located between the buttocks of a patient at or near where hemorrhoids may occur. Such treatment may often be preferred to surgery, the use of rectal inserts, or ointments. A thermal device of this kind can generally however only be used when a patient is entirely stationary.

### SUMMARY OF THE INVENTION

The invention provides a wearable hemorrhoidal aid which includes a waistband, a crotch portion, and a rear portion. The waistband forms a closed loop. The crotch portion has a front edge attached to a front section of the waistband. The rear portion has a front edge attached to a rear edge of the crotch portion and a rear edge attached to a rear edge of the waistband. The rear portion includes a pouch defining an enclosure. The pouch has an opening for insertion of a thermal insert into the enclosure. The thermal insert is also removable from the pouch.

### BRIEF DESCRIPTION OF THE DRAWINGS

The invention is further described by way of example with reference to the accompanying drawings wherein:

FIG. 1 is a top plan view of a wearable hemorrhoidal aid according to an embodiment of the invention;

FIG. 2 is a view from below of a pouch and an insert forming part of the wearable hemorrhoidal aid; and

FIG. 3 is a view from below, which is cross-sectioned to show internal components of the pouch and the insert.

### DETAILED DESCRIPTION OF THE INVENTION

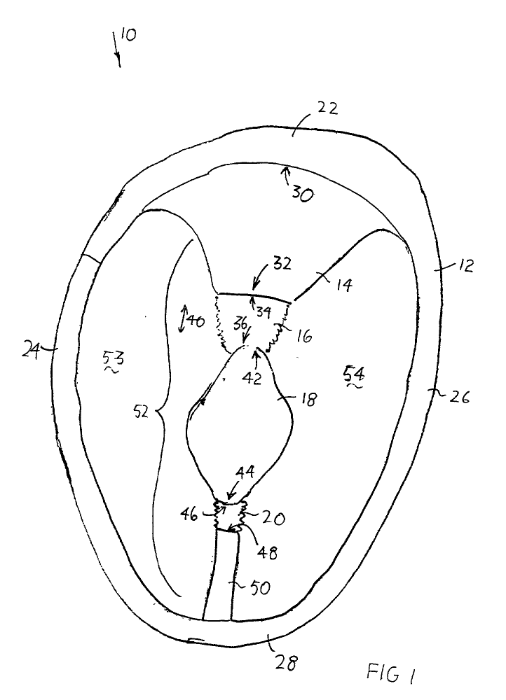

FIG. 1 of the accompanying drawings illustrates a wearable hemorrhoidal aid 10 according to an embodiment of the invention which includes a waist band 12, a crotch portion 14, a front elastic cover 16, a rear portion 18, and a rear elastic band 20.

The waistband 12 is formed in a closed loop and is typically made of an elastic material. The waistband 12 includes a front section 22, a left side section 24, a right side section 26, and a rear section 28.

The crotch portion 14 is typically made of a washable fabric such as cotton. The crotch portion 14 has a relatively wide front edge 30 and a relatively narrow rear edge 32. The entire front edge 30 is attached to the front section 22 of the waistband 12, typically by way of stitching.

The front elastic cover 16 has a front edge 34 and a rear edge 36. The front edge 34 is attached to the rear edge 32 of the crotch portion, typically by way of stitching. The material of the front elastic cover 16 is typically an elastic material that is washable. The front elastic cover 16 is stretchable in a direction 40, which causes movement of the edge 34 away from the edge 36, and in a direction transverse to the direction 40.

The rear portion 18 also has a front edge 42 and a rear edge 44. The front edge 42 is attached to the rear edge 36 of the front elastic cover 16, for example by way of stitching.

The rear elastic band 20 has a front edge 46 and a rear edge 48. The front edge 46 is attached to the rear edge 44 of the rear portion 18, for example by way of stitching. The rear edge 48 is attached to an intermediate section 50, also for example by way of stitching. The intermediate section 50 is attached to the rear section 28 of the elastic band 12. The rear elastic band 20 is made of a washable elastic material that can stretch in a direction 40.

A loop 52 is formed by the crotch portion 14, the front elastic cover 16, the rear portion 18, the rear elastic band 20, and the intermediate section 50. A front edge 30 of the loop 52 is secured to a front section 22 of the elastic band and a rear edge of the loop 52 is secured to a rear section 28 of the elastic band 12. A left leg opening 53 is formed between the left section 24 of the elastic band 12 and the loop 52 and a right leg opening 54 is formed between the right section 26 of the elastic band 12 and the loop 52.

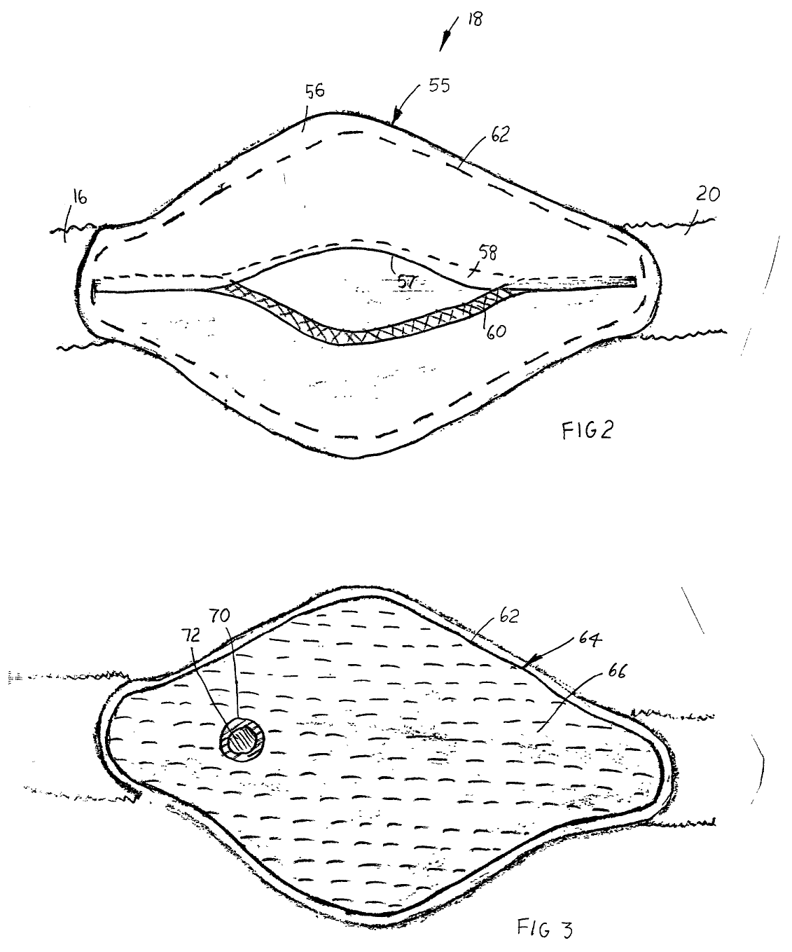

FIG. 2 illustrates the rear portion 18 in more detail. The rear portion 18 includes a pouch 55 that defines an enclosure 56. The pouch 55 is typically made of a soft non-allergenic washable rubber-like material. An elongate slit opening 57 is formed in one side of the pouch 55. The slit opening 57 extends from the front elastic cover 16 to the rear elastic band 20.

The wearable hemorrhoidal aid 10 further includes an interengaging strips 58 and 60 of Velcro®. In another embodiment other elongate members may be used such as interlocking strips of a zipper. The strips 58 and 60 are attached to opposing elongate edges of the slit opening 57.

As shown in FIG. 3, the wearable hemorrhoidal aid 10 may further include a thermal insert 62. The insert 62 includes a shell 64 and a thermal substance 66 located in the shell 64.

In one embodiment, the insert 62 may be located in a microwave and be microwaved to cause heating of the thermal substance 66. The thermal substance 66 may for example be heated to about 40° C. without destruction of shell 64. The insert 62 may alternatively be located in a freezer. The substance 66 may be a liquid at room temperature and freeze at a temperature of, for example, −10° C. In such an embodiment the insert 62 can be heated in a microwave or cooled in a freezer to temperatures above or below room temperature, as required.

In an alternative embodiment an actuator such as a capsule 70 having a reactive substance 72 therein may be located within the thermal substance 66. The capsule 70 is sufficiently thin so as to be easily ruptured when a person applies pressure to the shell 64 of the insert 62. Rupturing of the capsule 70 causes mixing of the reactive substance 72 with the thermal substance 66. Mixing of the reactive substance 72 with the thermal substance 66 may cause either heating or cooling of the thermal substance 66. The thermal substance 66 may for example be heated to a temperature above 40° C. or be cooled to a temperature below 5° C. Thermal and reactive substances of this kind are known in the art.

In use, the insert 62 may be heated or cooled as hereinbefore described. Referring to FIG. 2, a person may separate the opposing strips 58 and 60 and part the slit opening 57. The person may locate the heated or cooled insert 62 through the slit opening 57 into the enclosure 56. The insert 62 is only slightly smaller than the pouch 55 and has outer dimensions which closely match inner dimensions of the enclosure 56 so that the insert 62 fits snuggly within the enclosure 56. The person may then again attach the opposing strips 58 and 60 to one another, thereby ensuring that the insert 62 is retained within the enclosure 56. Heat is transferred from the insert 62 to the pouch 55 so that an outer surface of the pouch 55 is then at substantially the same temperature as the insert 62.

Referring to FIG. 1, a patient may insert his or her left leg through the left leg opening 53 and his or her right leg through the right leg opening 54. The patient pulls the waistband 12 up until it is located around a waist of the patient. Because the waistband 12 is made of an elastic material, the wearable hemorrhoidal aid 10 is retained in such a position. The crotch portion 14 is located over a crotch of the patient. The front elastic cover 16 may be dimensioned to fit over a vagina of a female or over a scrotum of a male. The pouch 55 and the insert 62 are located between the patient's buttocks. In such a position the pouch 55 can either heat or cool an area near the anus where hemorrhoids may occur. Stretching of the rear elastic band 20 allows for elongation of the loop 52 and for a small amount of adjustment in the positioning of the pouch 55.

The opening 57 is located on a side of the pouch 55 opposing an anal area of the person so as not to be irritating to the person. The person may wear the hemorrhoidal aid 10 while standing without danger that the pouch 55 may slip out. Because the strips 58 and 60 interengage with one another, the insert 62 is prevented from falling out of the enclosure 56.

While certain exemplary embodiments have been described and shown in the accompanying drawings, it is to be understood that such embodiments are merely illustrative and not restrictive of the current invention, and that this invention is not restricted to the specific constructions and arrangements shown and described since modifications may occur to those ordinarily skilled in the art.

What is claimed:

1. A wearable hemorrhoidal aid comprising:
a waist band forming a closed loop;
a crotch portion having a front edge attached to a front section of the waist band; and
a rear portion having a front edge attached to a rear edge of the crotch portion and a rear edge attached to a rear edge of the waist band, the rear portion including a pouch defining an enclosure and having an opening for insertion of a thermal insert into the enclosure, the thermal insert being removable from the pouch.

2. The wearable hemorrhoidal aid of claim 1 further comprising:
a front elastic cover securing the crotch portion to the rear portion.

3. The wearable hemorrhoidal aid of claim 1 further comprising:
a rear elastic band securing the rear portion to the rear section of the waistband.

4. The wearable hemorrhoidal aid of claim 1 wherein the opening is an elongate slit opening in a side of the pouch.

5. The wearable hemorrhoidal aid of claim 4 further comprising:
first and second elongate members attached to first and second opposing sides of the opening, the members being attachable to one another to close the opening and detachable from one another to open the opening.

6. The wearable hemorrhoidal aid of claim 5 wherein the elongate members are interlocking strips of a zipper.

7. The wearable hemorrhoidal aid of claim 5 wherein the elongate members are interengaging strips of Velcro®.

8. The wearable hemorrhoidal aid of claim 1 wherein the pouch is made of a soft non-allergenic washable material.

9. The wearable hemorrhoidal aid of claim 1 further comprising:
a thermal insert located in the enclosure defined in the pouch.

10. The wearable hemorrhoidal aid of claim 9 wherein the thermal insert includes a shell and a thermal substance located in the shell.

11. The wearable hemorrhoidal aid of claim 9 wherein microwaving of the insert causes heating of the substance to at least 40° C. without destruction of the shell.

12. The wearable hemorrhoidal aid of claim 8 further comprising:
an actuator in communication with the thermal substance and actuable to alter a temperature of the substance.

13. The wearable hemorrhoidal aid of claim 12 wherein the actuator is a capsule with a reactive substance therein which, when the capsule ruptures, mixes with the thermal substance and alters a temperature of the thermal substance.

14. The wearable hemorrhoidal aid of claim 13 wherein the temperature increases when the substances are mixed.

15. The wearable hemorrhoidal aid of claim 13 wherein the temperature decreases when the substances are mixed.

16. A wearable hemorrhoidal aid comprising:
a waist band forming a closed loop;
a crotch portion having a front edge attached to a front section of the waist band;
a rear portion having a front edge attached to a rear edge of the crotch portion and a rear edge attached to a rear edge of the waist band, the rear portion including a pouch defining an enclosure and having an opening;
a thermal insert inserted through the opening into the enclosure, the thermal insert including a shell and a thermal substance located in the shell;
a rupturable capsule; and
a reactive substance in the capsule which, when the capsule ruptures, mixes with the thermal substance and alters a temperature of the thermal substance.

17. The wearable hemorrhoidal aid of claim 16 wherein the temperature increases when the substances are mixed.

18. The wearable hemorrhoidal aid of claim 16 wherein the temperature decreases when the substances are mixed.
